(19) United States
(12) Patent Application Publication
(10) Pub. No.: US 2025/0268686 A1
(43) Pub. Date: Aug. 28, 2025

(54) APPLICATOR DEVICE, SYSTEM, AND METHOD FOR APPLYING SURGICAL SITE MARKERS

(71) Applicant: Vector Surgical, LLC, Waukesha, WI (US)

(72) Inventors: Janet L. F. Phillips, Nashotah, WI (US); Amanda Katsma, West Bend, WI (US); Melany Martha Wagner, Milwaukee, WI (US); Derek Roberts, Durham, NC (US)

(21) Appl. No.: 19/060,444

(22) Filed: Feb. 21, 2025

Related U.S. Application Data
(60) Provisional application No. 63/556,957, filed on Feb. 23, 2024.

(51) Int. Cl. A61B 90/00

(52) U.S. Cl. CPC A61B 90/39; A61B 2090/3937; A61B 2090/3966; A61B 2090/3987

(57) ABSTRACT

The disclosure relates to a surgical device for deploying site markers within a surgical site. The surgical device can include a housing, a carousel rotatably coupled to the housing, at least one site marker retained by the carousel, and a shaft assembly that extends through the housing and slidingly engages the housing and carousel, and a sheath coupled to the housing. The shaft assembly can include a drive member configured to engage the site marker and a retention member configured to selectively retain site marker. The sheath can be disposed around the shaft assembly and can include an opening at a distal end sized and shaped to permit the site marker to exit the surgical device.

### CLAIM OF PRIORITY

This application claims priority to U.S. Provisional Application No. 63/556,957, the disclosure of which is incorporated by reference herein in its entirety.

### FIELD

The present disclosure generally relates to a device for applying markers in a surgical site.

### BACKGROUND

The goal of tissue conserving cancer surgery is to remove the cancerous tissue while retaining as much healthy tissue as possible. Each year, over 400,000 cancer patients in the United States receive post-surgical radiation therapy as an adjunct to surgical resection of the cancerous tissue. While radiation therapy can be effective in reducing local recurrence rates of cancer, it is often accompanied by late-onset adverse effects. The primary objective of radiation therapy is to eliminate residual malignant cells within the surgical cavity. Yet current methods for identifying the boundaries of the surgical cavity remain imprecise, which can cause surrounding healthy tissues to be exposed to radiation in an effort to ensure that the entire surgical cavity is treated.

One method for marking surgical cavity boundaries involves the placement of small vessel clips, such as Hemoclips®. These clips, however, are used throughout surgery, generally for the purpose of closing blood vessels. If the clips are also used to delineate the boundaries or margins of a surgical site, the clip placement can be confusing when viewed by, for example, a different clinician at another time, or by a surgeon during a second surgery. The potential for confusion is augmented in that standard vessel clips typically lack any unique or differentiating feature which might indicate a specific margin or point of interest. These clips also tend to migrate around a surgical cavity because they do not reliably and securely attach to tissue, which was not their original purpose. Other types of clips for identifying surgical cavity boundaries present disadvantages as well. For instance, some clips may not be visible on different types of scans so as to clearly delineate the boundaries of a surgical site or cavity. Others are designed such that a single unit is to be used to articulate the entire surgical cavity, rather than multiple differentiated units that can articulate the unique size and shape of the entire surgical cavity. Other unsatisfactory clips are sometimes palpable outside the body, which can be upsetting to the patient. Some markers require suture to be attached to the tissue, which can be slow and inefficient and create potential for additional complications.

Given the foregoing, there is a need for an improved surgical site marker system for clearly and reliably identifying surgical cavity boundaries or margin planes.

### SUMMARY

The present disclosure relates to a device, system, and method for applying or deploying surgical site markers. In one aspect, the disclosed technology relates to a system for deploying one or more site markers within a surgical site. The system can include at least one site marker and an applicator device. The applicator device can include a housing, a carousel rotatably coupled to the housing and configured to retain the at least one site marker, a shaft assembly that extends through the housing and slidingly engages the housing and the carousel. The shaft assembly can include a drive member configured to engage the at least one site marker, and a retention member configured to selectively retain the at least one site marker. The applicator device can further include a sheath coupled to the housing. The sheath can be disposed around the shaft assembly and include a distal end having an opening sized and shaped to permit the at least one site marker to exit the applicator device.

The applicator device can be moveable between a retracted state and a deployment state by retracting or advancing the drive member. In some embodiments, the shaft assembly further comprises a spring-loaded guide member. In order to move the applicator device from the retracted state to the deployment state, the drive member can be configured to push the cavity marker out of the carousel and along a length of the shaft assembly, the site marker can be configured to engage with the retention member, and at least a portion of the at least one site marker can be disposed between the drive member and the guide member. When in the retracted state, the drive member can be retracted in a proximal direction such that it is capable of sufficiently disengaging the carousel to permit rotation of the carousel.

In some embodiments, the drive member further comprises a wedge portion, and when in the deployment state, the wedge portion can be configured to engage the retention member and cause the retention member to release one site marker. The carousel can retain a plurality of the site markers; and when in the retracted state, the carousel can be rotatable to permit selection of one of the plurality of site markers. The carousel can further include a plurality of windows corresponding to the plurality of site markers, whereby the site markers are visible through the corresponding windows while the site markers are in the carousel. When in the deployment state, the retention member can be configured to contact a stop of the sheath, wherein the stop prevents motion of the retention member relative to the sheath in a distal direction biased by a spring. In some embodiments, the spring is integrally formed with a flexible element of the retention member.

In some embodiments, a proximal end of the sheath can be coupled to a distal end of the housing, and the distal end of the sheath further includes a first deflection surface configured to contact a first arm of the site marker and bend the site marker such that the first arm moves closer to a second arm of the site marker. The deflection surface comprises an arcuate finger configured to bend the first arm of the site marker such that a gap is maintained between proximal portions of the first arm and second arm, while proximal and distal portions of the first arm and second arm move closer together. The distal end of the sheath can further include a second deflection surface configured to contact the second arm of the site marker, and the first and second deflection surfaces can be configured to bend the site marker simultaneously. The first and second deflection surfaces can be configured to bend the site marker when the site marker is moved distally into the first and second deflection surfaces. The site marker can be scan-detectable. For example, the site marker can be radiopaque.

In another aspect, the disclosed technology relates to an applicator device for applying a surgical site marker including a housing, a carousel rotatably coupled to the housing and configured to retain at least one site marker, a shaft assembly that extends through the housing and slidingly engages the housing and carousel, the shaft assembly including: a drive member configured to engage the site marker, and a retention member configured to selectively retain the site marker; and a sheath coupled to the housing, disposed around the shaft assembly, and comprising an opening at a distal end sized and shaped to permit the site marker to exit the applicator device.

In another aspect, the disclosed technology relates to a method of applying a site marker to tissue of a patient, the method including providing a surgical site marker system including a plurality of surgical site markers, and an applicator device. The applicator device can include: a housing, a carousel rotatably coupled to the housing, wherein the plurality of site markers are retained within the carousel, and a shaft assembly that extends through the housing and slidingly engages the housing and carousel, the shaft assembly including a drive member configured to engage one of the plurality of site markers. The method can further include retracting the drive member of the applicator device, rotating the carousel to select one of the plurality of site markers, inserting the delivery system into the patient proximate an area of tissue in a surgical site, and advancing the drive member distally to engage the selected site marker and secure the site marker to the area of tissue.

### BRIEF DESCRIPTION OF THE DRAWINGS

The accompanying drawings, which are incorporated herein and constitute part of this specification, are illustrative of particular embodiments of the present disclosure and do not limit the scope of the present disclosure. The drawings are not to scale and are intended for use in conjunction with the explanations in the following detailed description.

FIG. 1 is a perspective view of an example applicator device in an advanced state.

FIG. 2A is a perspective view of an example site marker.

FIG. 2B is a partial perspective top cross-sectional view of an example carousel of an applicator device.

FIG. 3A is a partial rear perspective cross-sectional view of an example applicator device.

FIG. 3B is a rear cross-sectional view of the example applicator device of FIG. 3A.

FIG. 3C is a perspective view of an example carousel of an applicator device.

FIG. 4A is a partial side cross-sectional view of an example applicator device.

FIG. 4B is another partial side cross-sectional view of an example applicator device.

FIG. 5 is a perspective view of an example applicator device in a retracted state.

FIG. 6 is a partial cross-sectional perspective view of the example applicator device of FIG. 5 in a retracted state.

FIG. 7 is another partial cross-sectional perspective view of the example applicator device of FIG. 5 in a retracted state.

FIG. 8 is a perspective view of an example retention member.

FIG. 9 is a partial cross-sectional perspective view of an example applicator device.

FIG. 10 is a perspective view of a distal end of an example applicator device in a retracted state.

FIG. 11 is a perspective view of a distal end of an example applicator device in a partially advanced state.

FIG. 12 is another perspective view of a distal end of an example applicator device in a partially advanced state.

FIG. 13 is partial cross-sectional perspective view a side view of an example applicator device in a partially advanced state.

FIG. 14 is a perspective view of a distal end of an example applicator device in a fully advanced state.

FIG. 15 is a perspective view of a distal end of an example applicator device in a partially advanced state.

### DETAILED DESCRIPTION

The present disclosure relates to a device, system, and method for applying or deploying surgical site markers. Surgical site markers are useful for delineating the boundaries, exterior surfaces, margins, areas of concern, or other features of a surgical site in a patient—e.g., a mammal such as a human. The site markers can be designed to clamp around tissue, pierce and clamp tissue, pinch tissue, or otherwise attached to tissue or otherwise pinch tissue within or near a surgical cavity. The markers can be designed to remain within the body after surgery or to mark tissue within the body that is to be excised or is already excised. Such markers that remain in the body can be used to: locate the surgical cavity margins for post-surgical treatment such as but not limited to radiation therapy; direct subsequent surgeries to a particular cavity margin if an excised specimen is positive for cancer based on pathology analysis or x-ray; locate the surgical margin that matches a positive specimen margin for more vigilant long-term post surgical surveillance to detect cancer recurrence, and other clinical post-surgical treatments and considerations. internally retained markers are especially useful in relation to surgeries that remove cancerous cells because the markers can later indicate, e.g., through additional scans, whether the surgical site was large enough to remove all of the cancerous cells. The disclosed site markers are designed to be visible on various types of scans (e.g., CT, X-ray, MRI, etc.) and to provide visual indicators of different portions of a surgical site. Examples of suitable site markers are described in WO2020106634.

Site markers can include two arms extending from a distal end of a base. The distal ends of the two arms can include points to engage tissue of an area of the surgical cavity. The site marker can be bent such that the two points move towards each other and grab the tissue between them. In some cases, one or both of the points may pierce the tissue. Extending from the proximal end of the base of the site marker is a marker indicator or indicium. The indicia can take a variety of forms including shapes, letters, numbers, symbols, etc. For example, indicia can take the form of letters, such as A, P, M, L, S, and I. The chosen letters can correspond to certain meanings or margin planes (e.g., A-anterior; P-posterior; M-medial; L-lateral; S-superior; I-inferior). Other indicia can correspond to other meanings or regions, such as radial, ulnar, proximal, distal, superficial, deep, cranial, caudal, canthal, dorsal, planter, ventral, endometrial, cervical, apex, base, right, left, retroperitoneal, costal, or clock positions (e.g., 3, 6, 9, 12 o'clock, etc.). Similarly, an indicium may represent an area of concern by the surgeon, to direct the focus for pathology or other post-surgical analysis, such as the location of a bone, nerve, or other vital anatomy which prevented the surgeon from excising additional tissue. Some symbols may be unspecified and assigned meaning by the surgeon.

In some embodiments, a site marker can be produced from one material in one piece. In some embodiments, the site marker can be radiopaque, or otherwise visible or detectable through various type of imaging techniques including but not limited to CT, X-ray, and MRI scans. As used herein, a scan-detectable site marker is one that is visible using a medical imaging technique. Site markers can be made from suitable materials, such as titanium, titanium alloys, carbon fiber or others. Preferably, the site markers are made of a material that is non-bioreactive.

The applicator device can include a housing, a sheath or tubular member, a carousel, and a plunger handle. The applicator device can further include housing handles on one or both sides. In general, the applicator device is moveable by a user between a retracted state and an advanced deployment state by moving the plunger handle. Retracting the plunger handle can permit selection of a site marker within the carousel by rotating the carousel. Subsequent depression of the plunger handle strips the site marker out of the carousel and moves it down the tubular member to a distal end of the applicator device. At this distal end, the site marker engages with a crimping portion that bends the distal-most end points of the site marker together, causing the site marker to grip tissue placed between the points, or otherwise attach to tissue. Preferably, the applicator device is made of a material that is non-bioreactive. A site marker applicator system can include one or more site markers and an applicator device.

The disclosed technology is next described with reference to the figures which provide examples of various embodiments. The use of these and other examples anywhere in the specification is illustrative only, and in no way limits the scope and meaning of the invention or of any exemplified form.

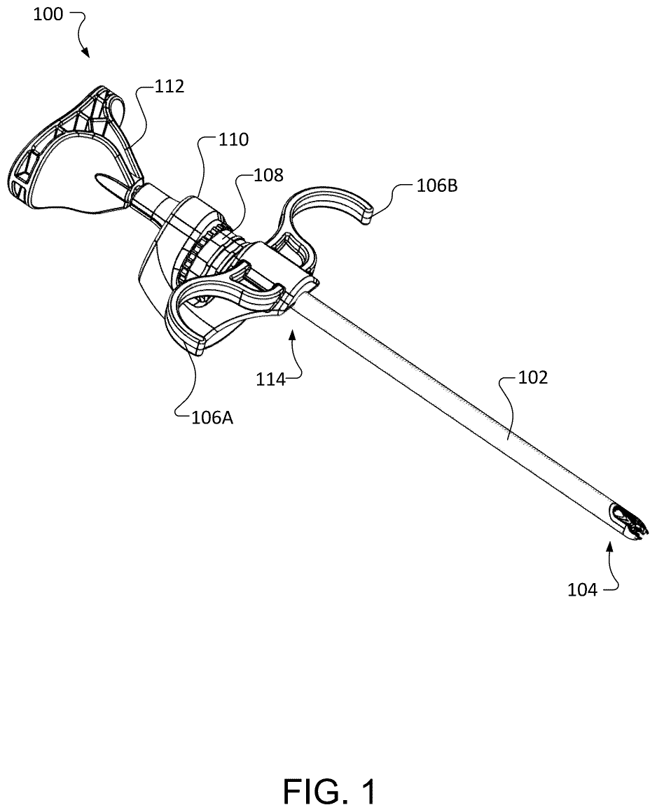

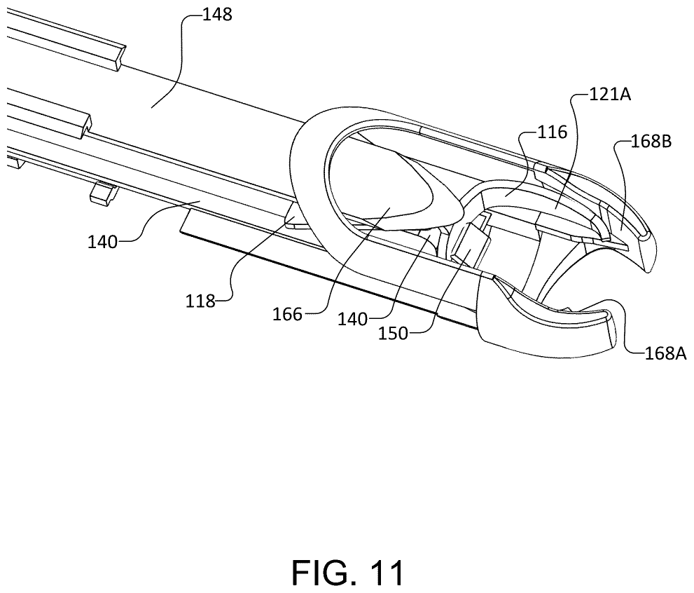

FIG. 1 is a perspective view of an applicator device 100. The applicator device can include a housing 110, a sheath or tube 102, a carousel 108, and a plunger handle 112. The various components of applicator device 100 can be constructed of medical grade plastics, stainless steel, or other suitable non-bioreactive materials. In some embodiments, various components such as those that contact other components and move relative to the other components, may have lubricious properties to limit wear of the parts from the movement. Applicator device 100 can further include housing handles 106A, 106B. Tube 102 can include a proximal end 114 and a distal end 104. The proximal end 114 can be coupled to housing 110. FIG. 1 illustrates applicator device 100 in an advanced position where the plunger handle 112 is advanced distally. The advanced position is explained in further detail with respect to FIGS. 11 to 15. In some embodiments, the overall length of the device 100 in the advanced position can be about 150 mm to 220 mm. In other embodiments, shorter or longer lengths are possible.

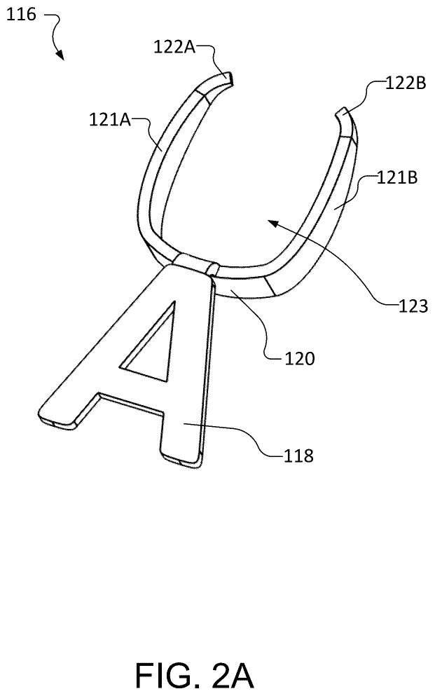

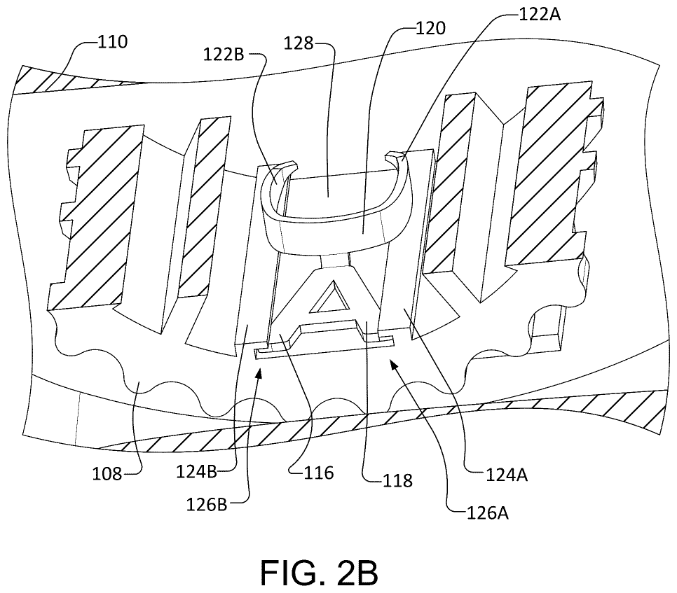

FIG. 2 is a perspective view of an example site marker 116. Site marker 116 can include a body 120, an indicia 118, and arms 121A, 121B. Arms 121A, 121B can include distal ends 122A, 122B respectively. Distal ends 122A, 122B can include points (e.g., for penetrating or gripping tissue around a surgical site). Site marker 116 can further include a gap 123, which can be maintained when the site marker 116 is applied to a surgical site. This gap 123 allow secure attachment of the clip to tissue, without site marker 116 crushing or damaging tissue or constricting blood supply. Indicia 118 can take a variety of forms. As illustrated in FIG. 2A, indicia 118 is a capital letter “A.” Other forms, such as shapes, letters, numbers, symbols, or others are possible. For example, indicia can take the form of the letters A, P, M, L, S, and I, which can correspond to certain meanings (e.g., A—anterior; P—posterior; M—medial; L—lateral; S—superior; I—inferior). Other indicia can correspond to various meanings or regions, such as radial, ulnar, proximal, distal, superficial, deep, cranial, caudal, canthal, dorsal, planter, ventral, endometrial, cervical, apex, base, right, left, retroperitoneal, costal, or all clock faces (esp. 3, 6, 9, 12 o'clock). Site markers 116 can be made in a variety of sizes for specific applications. For example, the distance between distal ends 122A, 122B can be between about 0.5 mm to about 6 mm or larger, or between about 2 mm and about 3 mm. As another example, the overall length of the site markers 116 can be between about 5 mm to about 12 mm, or about 6 mm to about 10 mm, or about 7 mm to about 9 mm. Similarly, as an example, the overall width of site markers 116 can be about 4 mm, or about 3 mm to about 5 mm, or about 2 mm to about 6 mm.

Site markers 116 can be retained in carousel 108 of applicator device 100. Carousel 108 can be rotatable coupled to the housing 110 of the applicator device 100, which allows a user to rotate the carousel to different positions to select a particular site marker 116. FIG. 2B is a partial cross-sectional view of carousel 108 within housing 110. FIG. 2B illustrates placement and retention of site marker 116 in carousel 108. Specifically, as depicted, carousel 108 can retain the indicia portion 118 of site marker 116. Outer edges of indicia 118 can be retained in grooves 126A, 126B (i.e., between surface 128 and ridges 124A, 124B). Grooves 126A, 126B can run longitudinally (i.e., substantially parallel to the length of sheath 102 and drive member 140). This orientation permits the site marker 116 to be pushed from through grooves 116 to release the site marker 116 from carousel 108 during deployment. As such, indicia 118 can ride along surface 128 until it is release from carousel 108. Indicia 118 can be retained between surface 128 and ridges 124A, 124B.

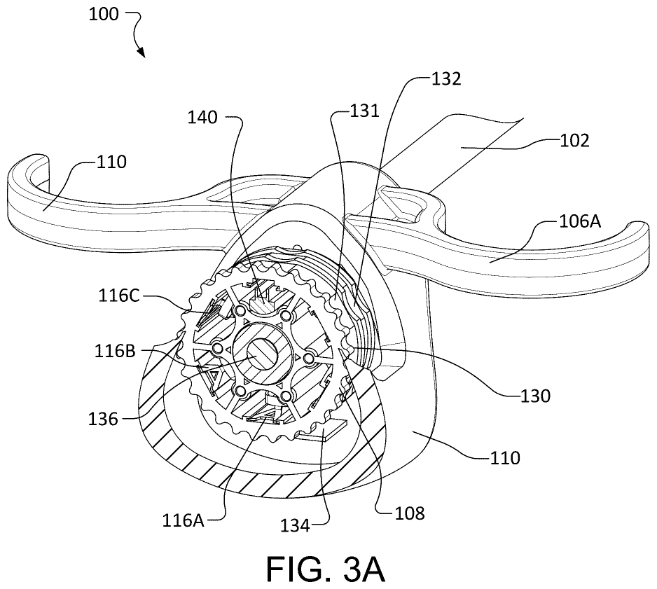

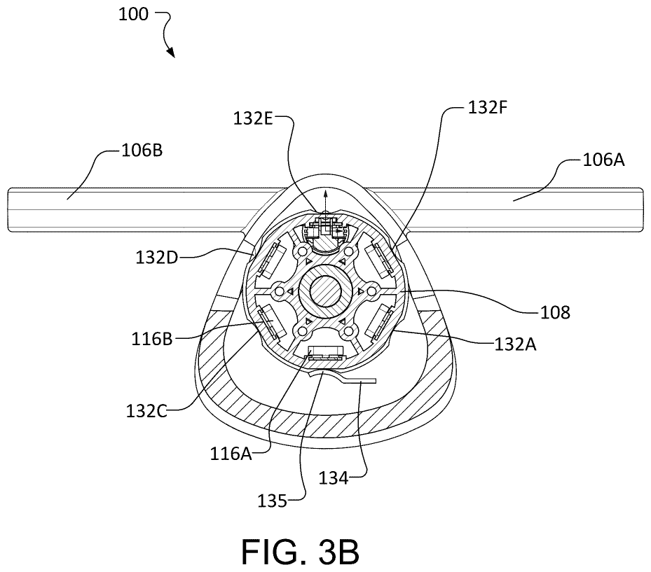

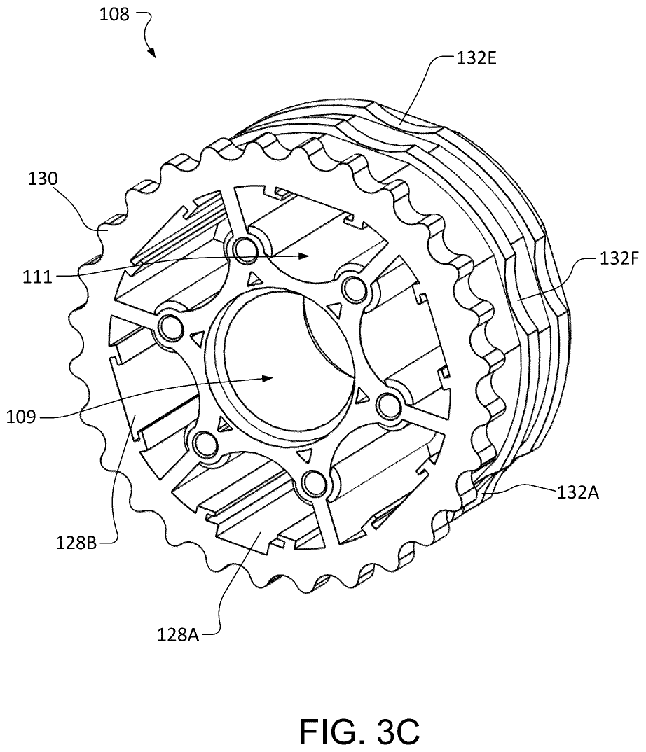

FIG. 3A is a partial rear perspective cross-sectional view of applicator device 100. FIG. 3B is a rear cross-sectional view of applicator device 100. In the illustrated embodiment, carousel 108 can house six separate site markers 116. However, carousel 108 can be configured to retain more or fewer site markers 116. For example, a relatively larger carousel could retain more site markers. As a more specific example, carousel 108 could retain 1, 2, 3, 4, 5, 6, 7, 8, 9, 10, 11, or 12 site markers. The dimensions of carousel 108 can be configured depending on the number of site markers retained, the size of the site markers, etc. In the illustrated example of carousel 108 housing 6 site markers, carousel 108 can have an outer diameter of about 18 to 22 mm, but it is understood that other sizes are possible. Carousel 108 can be rotatably coupled to housing 110 via shaft 136. Shaft 136 can extend through aperture 109 of carousel 108. Additionally, carousel 108 can include one or more detents 132. Detents 132A-F can engage with locking mechanism 134. Locking mechanism 134 can include a tab 135 that engages with a detent and is configured to flex to disengage the detent when carousel 108 is rotated. Carousel 108 can include a detent corresponding to each of site markers 116. Accordingly, carousel 108 can be stably locked in position for deployment of any one of the site markers 116 retained by carousel 108. Locking mechanism 134 can take other forms such as, but not limited to, a spring-loaded ball detent mechanism.

Carousel 108 can further include ridges 130. Ridges 130 can aid a user in gripping carousel 108 to rotate it to select a site marker. Additionally, carousel 108 can include one or more windows (not pictured). The windows can be placed through the outer wall 131 of carousel 108 such that a user can look through outer wall 131 and view the indicia of the site marker located in the corresponding area of carousel 108. Accordingly, a user can load carousel 108 with multiple site markers having different indicia. The user can then rotate carousel 108 and use the windows to view the various indicia in order to select a specific site marker featuring the desired indicium to apply to a surgical site.

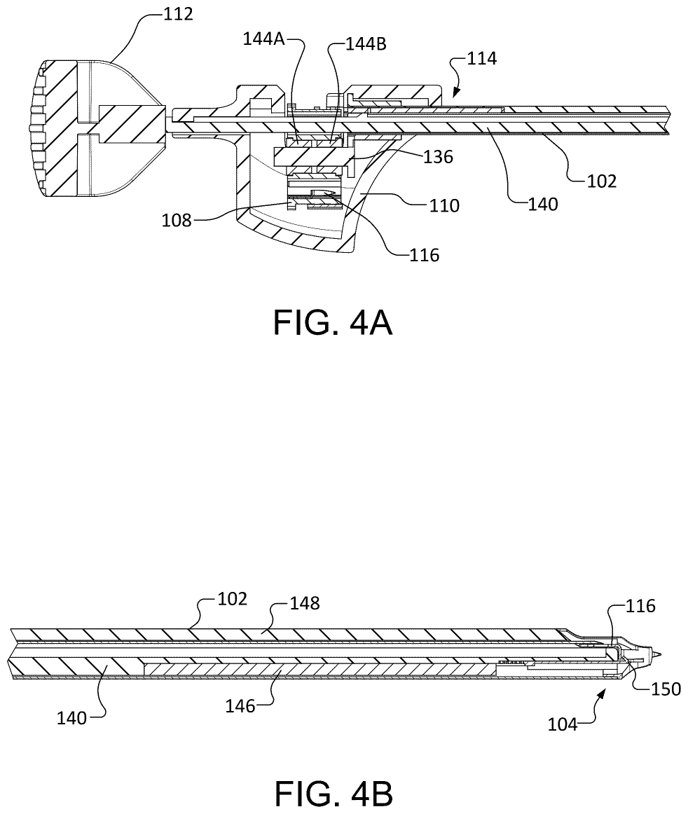

FIG. 4A is a side cross-sectional view of a proximal end portion of applicator device 100. FIG. 4B is a side cross-sectional view of a distal end portion of applicator device 100. As shown, a shaft assembly can be disposed within sheath 102. The shaft assembly can include a drive member 140, a rigid member 146, a guide member 148, and a retention member 150.

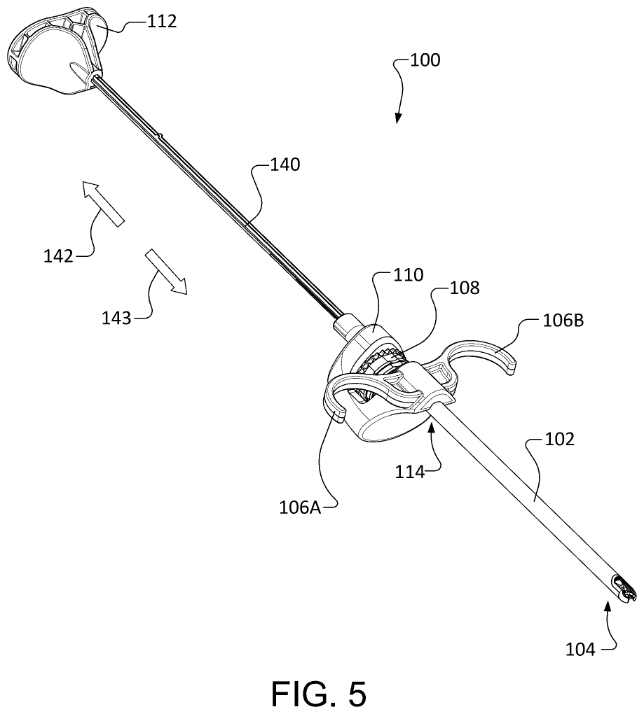

FIG. 5 is a perspective view of applicator device 100 in a retracted state. In the retracted state, the shaft assembly is retracted from sheath 102 and carousel 108. This permits rotation of carousel 108 within housing 110 to enable selection of a particular site marker. Applicator device 100 can enter the retracted state when a user retracts plunger handle 112 in a proximal direction (i.e., direction 142). By contract, applicator device 100 can enter the advanced state (i.e., the state for deployment of a site marker) when a user advances or pushes plunger handle 112 in a distal direction (direction 143). Put differently, the applicator device is moveable between the retracted state and the advanced (deployment) state by advancing or retracting the drive member.

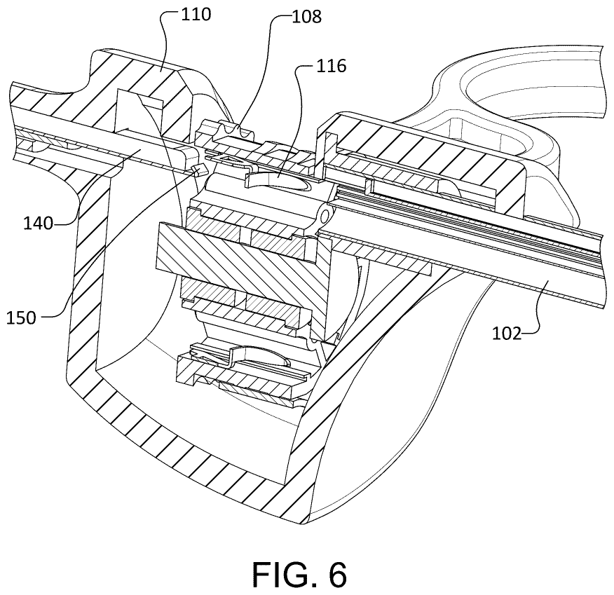

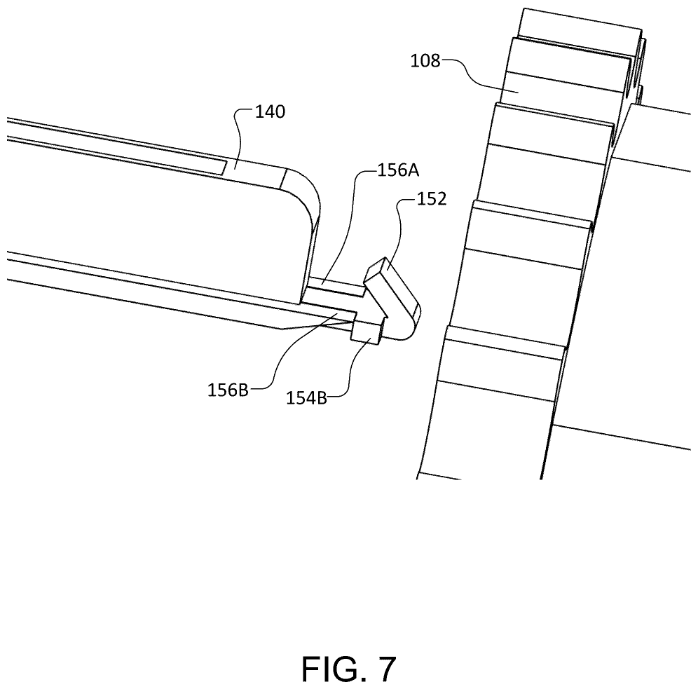

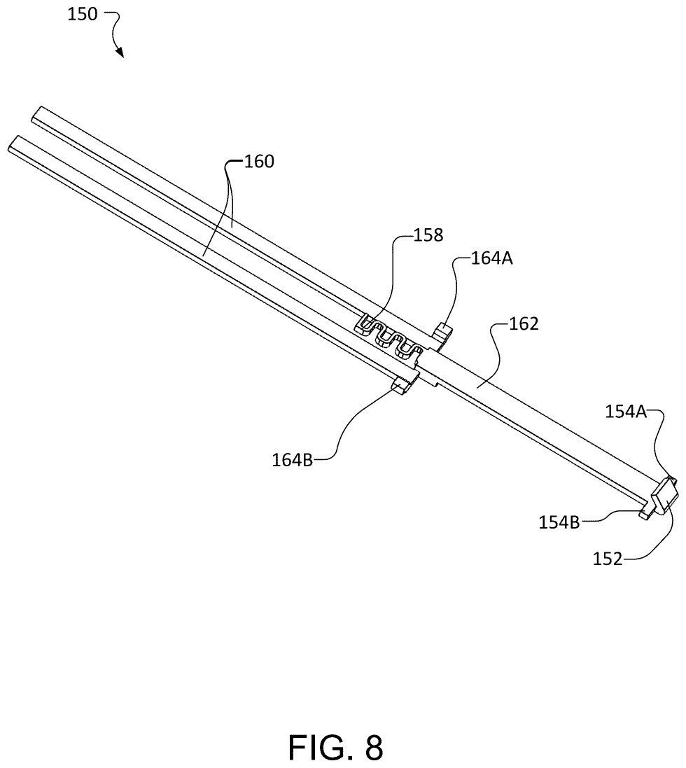

FIGS. 6 and 7 are partial cross-sectional perspective view of applicator device 100 in the retracted state. As illustrated, the drive member 140 and retention member 150 are retracted far enough to clear carousel 108. In other words, the drive member is retracted in a proximal direction such that it sufficiently disengages the carousel to permit rotation of the carousel. FIG. 8 is a perspective view of retention member 150.

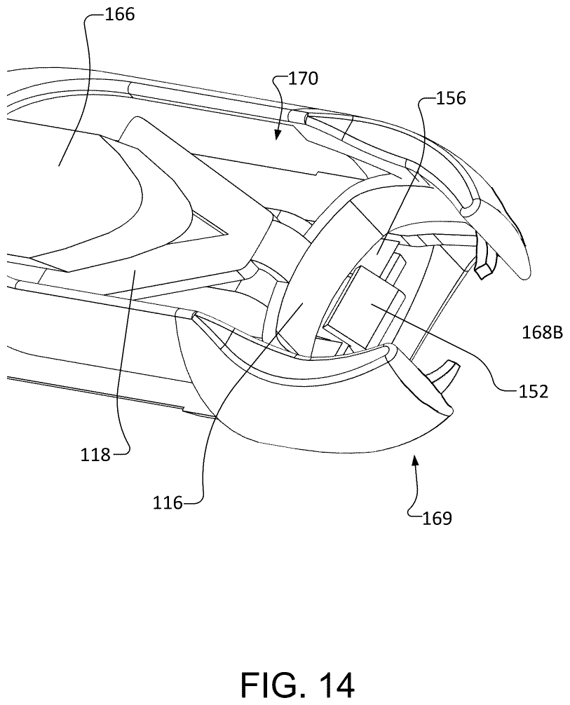

Referring to FIGS. 6-8, retention member 150 can include a protrusion 152. Protrusion 152 can permit retention member 150 to selectively retain a site marker. Protrusion 152 can be placed at an angle to flexible element 162 of retention member 150. In other words, protrusion 152 may be an angled protrusion. Protrusion 152 can be positioned at an angle of about 45 degrees to flexible element 162. In other embodiments, other angles are possible, for example between 20 degrees and 80 degrees. The angle can act as a wedge to drive protrusion 152 down and out of the way when the shaft assembly is engaging a site marker. Retention member 150 can further include release ledges 154A, 154B. Release ledges can be configured to engage with corresponding wedges 156A, 156B of drive member 140. When drive member 140 is advanced far enough relative to retention member 150, wedges 156A, 156B will contact release ledges 154A, 154B and drive protrusion 152 down, causing it to release an engaged site marker. This released position is illustrated in FIG. 14, which is described below.

Retention member 150 can further include a spring 158. Spring 158 can extend from body 162. In some embodiments, spring 158 can be integrally formed with retention member 150 as one piece. Retention member 150 can also include distal position stops 164A, 164B. Distal position stops 164A, 164B can be configured to prevent retention member 150 from traveling beyond a certain point in a distal direction. This permits drive member 140 to continue traveling distally relative to retention member 150 (thereby compressing spring 158). This continued relative motion permits wedges 156 to engage contact release ledges 154A, 154B and drive protrusion 152 down, thereby permitting release of a site marker. Retention member 150 can be made of a resilient material that can bend and spring back to its original position, such as a metal (for example, a suitable steel alloy), carbon fiber, or other suitable material.

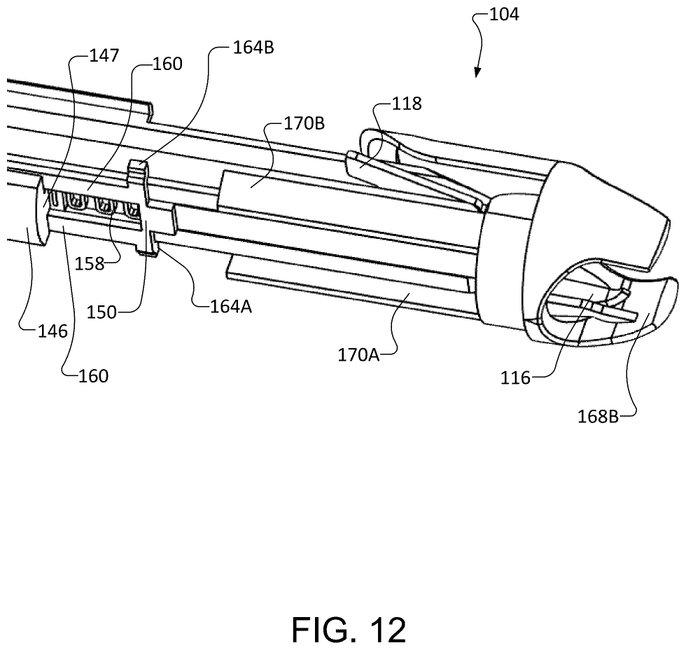

Retention member 150 can further include a pair of alignment features 160. Alignment features 160 can engage with the sides of sheath 102 or with rigid member 146 to keep retention member 150 axially aligned within sheath 102. Additionally, the gap between alignment features 160 can permit protrusion of rigid member 146 to engage spring 158. For example, see FIG. 12 which illustrates protrusion 147 of rigid member 146 contacting spring 158 between alignment features 160.

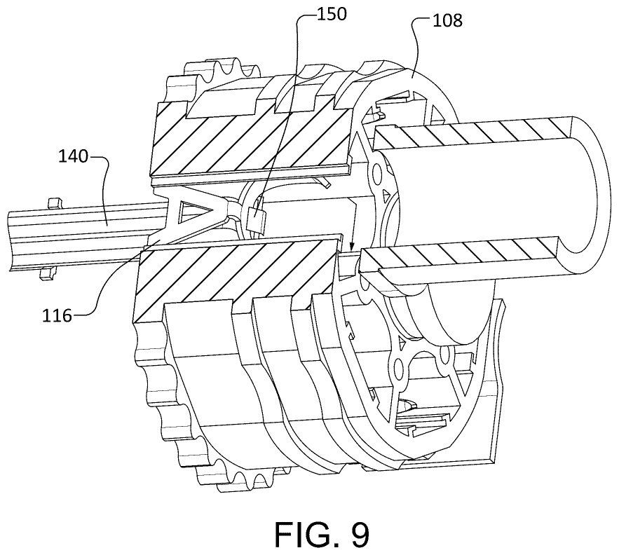

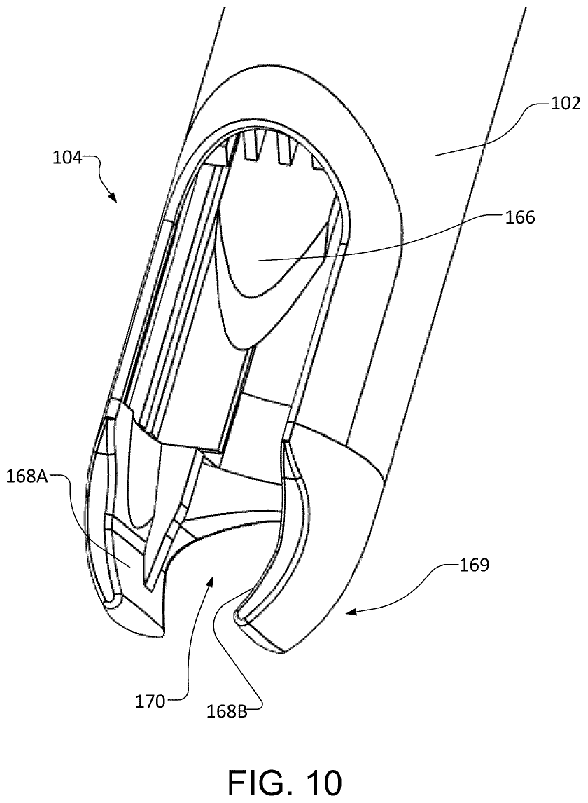

FIG. 9 is a partial cross-sectional perspective view of applicator device 100. As described above, the shaft assembly can slidingly engage the housing 110 and carousel 108. Specifically, drive member 140 can be configured to engage the site marker. When moved in a distal direction, angled protrusion 152 of retention element 150 can clip around site marker 116, as shown in FIG. 9, causing site maker 116 to be retained between drive member 140 and retention element 150.

FIG. 10 is a perspective view of a distal end 104 of an example applicator device 100 in a retracted state. Sheath 102 can include a distal end 104 having a crimping portion 169 and an opening 170. Opening 170 can be sized and shaped to permit a site marker to exit the applicator device 100 through opening 170. The crimping portion 169 can include one or more deflection surfaces 168A, 168B. Deflection surfaces 168A, 168B can be configured to contact arms of the site marker and bend them in towards one another to grip tissue. In other words, the first deflection surface 168B can be configured to contact a first arm 121A of the site marker 116 and bend the site marker such that the first arm 121A moves closer to a second arm 121B of the site marker 116.

The deflection surfaces 168A, 168B can take the form of arcuate fingers, as illustrated in the Figures. The arcuate fingers can be configured to bend the arms of a site marker in a curved fashion when the site marker is forced into the arcuate fingers by the drive member 140. The precise curvature of arcuate fingers can depend on the specific dimensions or other geometry of the site marker, or on the characteristics of the type of tissue to which the marker is designed to attach. The arcuate fingers provide the advantage of closing the distal portions 122A, 122B of site marker 116 without closing gap 123. Accordingly, the arcuate fingers can be configured to bend the corresponding arms of the site marker such that a gap is maintained between proximal portions of the first arm and second arm, while and distal portions of the first arm and second arm move closer together. This can prevent additional tissue damage, while also helping hold the site marker in place, thereby preventing migration of the site marker over time.

Crimping portion 169 can be formed from a rigid material, such as steel or carbon fiber. Crimping portion 169 must be sufficiently stiff to bend one or both arms 121A, 121B of a site marker in order for the applicator device to apply the site marker to a surgical site.

Guide member 148 can include distal end 166. Guide member 148 can be spring-loaded and retractable such that distal end 166 releases site marker 116 and permits site marker 116 to exit through opening 170.

To move from the retracted state to an advanced or deployment state, a user can push plunger handle 112 distally toward distal end 104. This will cause the shaft assembly to move in the same direction. Retention member 150 will engage the clip and drive member 140 will push the site marker out of the carousel 108 and down sheath 102. At least a portion of the site marker 116 will be retained by retention member 150 (i.e., the body 120 will be retained between retention member 150 and distal end of drive member 140). At least another portion of the site marker 116 can be held between guide member 148 and drive member 140. Eventually, site marker 116 will reach distal end 104.

FIGS. 11 and 12 are perspective views of distal end 104 of an example applicator device 100 in a partially advanced state where arms 121 of site marker 116 have just started to contact deflection surfaces 168. For illustration purposes, the tubular member of sheath 102 is not depicted in these figures. In this position, retention member 150 is still retaining site marker 116. Similarly, distal end 166 of guide member 148 is still retaining indicia 118. The gap between sheath stops 170A, 170B and retention element stops 164A, 164B indicates that the retention element 150 has not released site marker 116 and that further distal movement of the shaft assembly is possible.

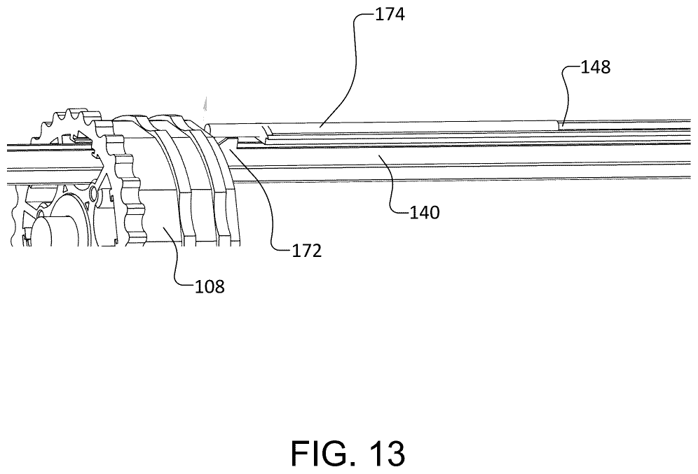

FIG. 13 is partial cross-sectional perspective view a side view of applicator device 100 in the partially advanced state. The guide member 148 can be spring loaded or biased in the distal direction by a spring 174. Spring 174 can be a compression spring that contacts a protrusion from sheath 102 (not illustrated in FIG. 13 for clarity) and pushes guide member 148 in the proximal direction (toward housing 110). Spring 174 will become further compressed as drive member 140 is advanced distally. When drive member 140 is retracted, the spring will pull guide member 148 proximally, thereby allowing distal end 166 of guide member 148 to release site marker 116 and may return guide member 148 to its original non-advanced position.

Referring to FIGS. 12 and 14, when retention element stops 164A, 164B contact sheath stops 170A, 170B, further movement of drive member 140 will push rigid member 146 into spring 158, causing it to compress. Compression of spring 158 will permit relative motion between drive member 140 and retention element 150 (which will in turn cause wedges 156 to contact release ledges 154A, 154B and drive protrusion 152 down, causing the site marker 116 to be released by retention element 150. This position is illustrated by FIG. 14. As such, the first and second deflection surfaces can be configured to bend the site marker simultaneously.

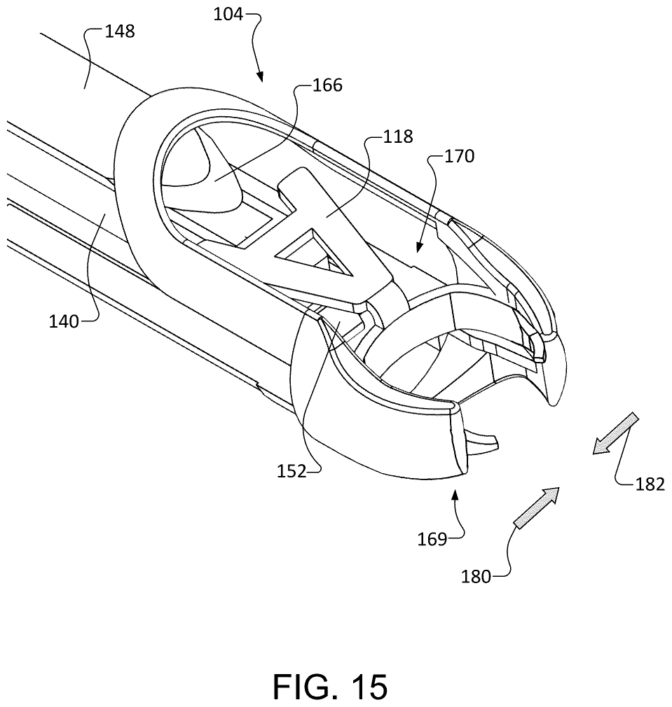

FIG. 15 is a perspective view of a distal end 104 of an example applicator device 100 in a partially advanced state. Once the drive member 140 has been advanced far enough that retention member 150 releases site marker 116, drive member 140 can be retracted. When drive member 140 is retracted proximally, distal end 166 of guide member 148 will retract due to the spring forces described above. This retraction of distal end 166 will completely release site marker 116 and permit site marker 116 to exit the device through opening 170, while remaining attached to tissue of the surgical site.

Disclosed embodiments further include a method of applying a site marker in a patient. The site marker can be one or more of a plurality of surgical site markers. The method can include providing a surgical site marker system that applies the one or more of the plurality surgical site markers. The system can be substantially as described above. For example, the system can include a plurality of surgical site markers and an applicator device. As described above, the applicator device can include a housing, a carousel rotatably coupled to the housing and configured to retain the plurality of site markers, and a shaft assembly that extends through the housing and slidingly engages the housing and carousel, the shaft assembly comprising a drive member configured to engage the site marker. The method can further include retracting the drive member of the applicator device. Retracting the drive member can include pulling on a plunger handle attached to the drive member and moving the drive member in a proximal direction until the distal end of the drive member clears the carousel. The method can further include rotating the carousel to select a desired site marker of the plurality of site markers. This step of the method can include viewing one or more of the plurality of site markers through a window or windows of the carousel. The method can further include inserting the delivery system into the patient proximate to an area of tissue of the surgical site. The selection of the desired site marker can, for example, be based on the area of tissue on which the user desires to place the site marker. The method can further include advancing the drive member distally to engage the desired site marker and apply the site marker to the area of tissue. Advancing the drive member distally can cause a retention member of the shaft assembly to engage the site marker. Further distal advancement of the drive member can cause the site marker to contact a deflection surface at the distal end of the applicator device, causing the site marker to pinch and engage the tissue. The further distal advancement can also cause the retention member to release the site applicator. The method can further include retracting the drive member again, which can cause the site marker to be fully released from the applicator device.

Certain relationships between features of the device are described herein using the term “substantially” or “substantially equal.” As used herein, the terms “substantially” and “substantially equal” indicate that the equal relationship is not a strict relationship and does not exclude functionally similar variations therefrom. Unless context or the description indicates otherwise, the use of the term “substantially” or “substantially equal” in connection with two or more described dimensions indicates that the equal relationship between the dimensions includes variations that, using mathematical and industrial principles accepted in the art (e.g., rounding, measurement or other systematic errors, manufacturing tolerances, etc.), would not vary the least significant digit of the dimensions. As used herein, the term “substantially parallel” indicates that the parallel relationship is not a strict relationship and does not exclude functionally similar variations therefrom. As used herein, the term “substantially orthogonal” indicates that the orthogonal relationship is not a strict relationship and does not exclude functionally similar variations therefrom.

The foregoing discussion merely illustrates the principles of the disclosure and omits or only briefly describes conventional features of the disclosed technology that are apparent to those skilled in the art. Reference to various embodiments does not limit the scope of the claims attached hereto. Additionally, any examples set forth in this specification are intended to be non-limiting and merely set forth some of the many possible embodiments for the appended claims. The invention is therefore to be limited only by the terms of the claims, along with the full scope of equivalents to which the claims are entitled. Further, particular features described herein can be used in combination with other described features in each of the various possible combinations and permutations. A person of ordinary skill in the art would know how to use the instant invention, in combination with routine experiments, to achieve other outcomes not specifically disclosed in the examples or the embodiments. Those skilled in the art will readily recognize various modifications and changes that may be made without following the example embodiments and applications illustrated and described herein, and without departing from the true spirit and scope of the following claims.

Unless otherwise specifically defined herein, all terms are to be given their broadest possible interpretation including meanings implied from the specification as well as meanings understood by those skilled in the art and/or as defined in dictionaries, treatises, etc. Unless defined otherwise, all technical and scientific terms used herein have the same meaning as commonly understood by one of ordinary skill in the art in the field of the disclosed technology. It must also be noted that, as used in the specification and the appended claims, the singular forms “a,” “an” and “the” include plural referents unless otherwise specified, and that the terms “includes” and/or “including,” when used in this specification, specify the presence of stated features, elements, and/or components, but do not preclude the presence or addition of one or more other features, steps, operations, elements, components, and/or groups thereof. Additionally, methods, equipment, and materials similar or equivalent to those described herein can also be used in the practice or testing of the disclosed technology.

All references cited and/or discussed in this specification are incorporated herein by reference in their entireties and to the same extent as if each reference was individually incorporated by reference.

What is claimed is:

1. A system for deploying one or more site markers within a surgical site, the system comprising:
at least one site marker; and
an applicator device comprising:
a housing;
a carousel rotatably coupled to the housing and configured to retain the at least one site marker;
a shaft assembly that extends through the housing and slidingly engages the housing and the carousel, wherein the shaft assembly comprises:
a drive member configured to engage the at least one site marker, and
a retention member configured to selectively retain the at least one site marker; and
a sheath coupled to the housing, wherein the sheath is disposed around the shaft assembly which comprises a distal end having an opening sized and shaped to permit the at least one site marker to exit the applicator device.

2. The system of claim 1, wherein the applicator device is moveable between a retracted state and a deployment state by retracting or advancing the drive member.

3. The system of claim 2, wherein the shaft assembly further comprises a spring-loaded guide member.

4. The system of claim 3, wherein, in order to move the applicator device from the retracted state to the deployment state:
the drive member is configured to push the cavity marker out of the carousel and along a length of the shaft assembly,
the site marker is configured to engage with the retention member; and
at least a portion of the at least one site marker is disposed between the drive member and the guide member.

5. The system of claim 2, wherein, when in the retracted state, the drive member is retracted in a proximal direction such that it is capable of sufficiently disengaging the carousel to permit rotation of the carousel.

6. The system of claim 2, wherein:
the drive member further comprises a wedge portion; and
when in the deployment state, the wedge portion is configured to engage the retention member and cause the retention member to release one site marker.

7. The system of claim 2, wherein:
the carousel retains a plurality of the site markers; and
when in the retracted state, the carousel is rotatable to permit selection of one of the plurality of site markers.

8. The system of claim 7, wherein the carousel further comprises a plurality of windows corresponding to the plurality of site markers, whereby the site markers are visible through the corresponding windows while the site markers are in the carousel.

9. The system of claim 2, wherein, when in the deployment state, the retention member is configured to contact a stop of the sheath, wherein the stop prevents motion of the retention member relative to the sheath in a distal direction biased by a spring.

10. The system of claim 9, wherein the spring is integrally formed with a flexible element of the retention member.

11. The system of claim 1, wherein:
a proximal end of the sheath is coupled to a distal end of the housing; and
the distal end of the sheath further comprises a first deflection surface configured to contact a first arm of the site marker and bend the site marker such that the first arm moves closer to a second arm of the site marker.

12. The system of claim 11, wherein the deflection surface comprises an arcuate finger configured to bend the first arm of the site marker such that a gap is maintained between proximal portions of the first arm and second arm, while proximal and distal portions of the first arm and second arm move closer together.

13. The system of claim 11, wherein:
the distal end of the sheath further comprises a second deflection surface configured to contact the second arm of the site marker; and
the first and second deflection surfaces are configured to bend the site marker simultaneously.

14. The system of claim 13, wherein: the first and second deflection surfaces are configured to bend the site marker when the site marker is moved distally into the first and second deflection surfaces.

15. The system of claim 1, wherein the site marker is scan-detectable.

16. The system of claim 15, wherein the site marker is radiopaque.

17. A applicator device for applying a surgical site marker comprising:
a housing;
a carousel rotatably coupled to the housing and configured to retain at least one site marker;
a shaft assembly that extends through the housing and slidingly engages the housing and carousel, the shaft assembly comprising:
a drive member configured to engage the site marker, and
a retention member configured to selectively retain the site marker; and
a sheath coupled to the housing, disposed around the shaft assembly, and comprising an opening at a distal end sized and shaped to permit the site marker to exit the applicator device.

18. A method of applying a site marker to tissue of a patient, the method comprising:
providing a surgical site marker system comprising:
a plurality of surgical site markers; and
an applicator device comprising:
a housing,
a carousel rotatably coupled to the housing, wherein the plurality of site markers are retained within the carousel, and
a shaft assembly that extends through the housing and slidingly engages the housing and carousel, the shaft assembly comprising a drive member configured to engage one of the plurality of site markers;
retracting the drive member of the applicator device;
rotating the carousel to select one of the plurality of site markers;
inserting the delivery system into the patient proximate an area of tissue in a surgical site; and
advancing the drive member distally to engage the selected site marker and secure the site marker to the area of tissue.
